(19) United States
(12) Patent Application Publication
(10) Pub. No.: US 2011/0117131 A1
(43) Pub. Date: May 19, 2011

(54) Production of OspA for Lyme Disease Control

(76) Inventors: Ning Huang, Davis, CA (US); Deshui Zhang, Woodland, CA (US); Somen Nandi, Sacramento, CA (US); Lyle Robert Petersen, Fort Collins, CO (US)

(21) Appl. No.: 12/936,499

(22) PCT Filed: Apr. 9, 2009
(86) PCT No.: PCT/US09/40083

§ 371 Date: Jan. 26, 2011

Related U.S. Application Data
(60) Provisional application No. 61/071,032, filed on Apr. 9, 2008.

(51) Int. Cl. A61K 39/02; A01H 5/00; A01H 5/10; C12P 21/00; C12N 15/63; A61P 37/04; A61P 31/04

(52) U.S. Cl. 424/234.1; 800/298; 800/320.2; 800/320.1; 800/320; 800/320.3; 435/69.1; 435/320.1

(57) ABSTRACT

The present invention relates, generally, to the production of one or more OspA proteins in plant cells. Heterologous DNA comprising genes encoding one or more desired OspA protein(s) are introduced into plant cells. The one or more OspA protein(s) can be recombinantly-produced in the plant cells, optionally purified from the plant cells, and used as an oral vaccine to prevent the transmission of Lyme disease, particularly by animal vectors. The recombinantly-produced OspA protein(s) can be provided in oral and parenteral formulations. The present invention also relates to oral administration of OspA protein(s) to vaccinate against Lyme disease. The OspA protein(s) may be provided in a dosage form that is suitable for oral administration as a vaccine to prevent an animal from developing Lyme disease after exposure to a source of Borrelia burgdorferi.

### RELATED APPLICATION DATA

This application claims priority to U.S. Provisional Application No. 61/071,032, filed Apr. 9, 2008. The contents of this application are incorporated herein by reference in their entirety.

### BACKGROUND OF THE INVENTION

1. Field of the Invention

The present invention relates, generally, to methods for the recombinant production of one or more OspA proteins in plant cells, where the resulting OspA protein(s) may be used, for example, in methods of orally vaccinating an animal against Lyme disease. The present invention further relates to recombinantly-produced OspA protein(s) provided in a form that is suitable for oral administration as a vaccine, in order to prevent an animal from developing Lyme disease after subsequent exposure to a source of Borrelia burgdorferi.

2. Description of the Related Art

Lyme disease is caused by a spiral-shaped bacterium, Borrelia burgdorferi, which is transmitted to humans by the bite of infected blacklegged ticks. These ticks find hosts in a variety of different wild animals.

The number of cases of Lyme disease caused by Borrelia burgdorferi has been steadily increasing, and it is a public health imperative to control the spread of this disease, which is difficult to diagnose and treat. The incidence of Lyme disease has increased to level of more than 25,000 cases per year in the United States. One strategy for controlling the spread of Lyme disease is to vaccinate wild animals known to harbor the ticks, thereby controlling the spread of Lyme disease.

U.S. Pat. No. 6,183,986 (Bergstrom et al.) describes the preparation of fractions from B. burgdorferi spirochetes, and the isolation and sequencing of the outer surface protein A (OspA) gene. The gene is said to be capable of use in a vaccine.

The outer surface protein A (OspA) expressed by B. burgdorferi in the tick mid-gut is an effective antigen, and humans and mice vaccinated with OspA proteins are well-protected from B. burgdorferi infection. (See Steere et al., N. Engl. J. Med. 339:209-15 (1998).) A vaccine to protect humans from Lyme Disease, called Lymerix, was previously-developed based on OspA of Borrelia. (See Sigal et al., N. Engl. J. Med. 339:216-22 (1998).) However, the vaccine was pulled from the market in 2002 for numerous reasons. Gomes-Solecki et al. describe an oral bait delivery system containing an OspA protein obtained from transformed E. coli. The oral vaccine protected 89% of mice, and resulted in an eight-fold reduction in the amount of B. burgdorferi present in tick vectors (Vaccine 24:4440-49 (2006)).

Thus, it would be advantageous to produce large amounts of an OspA protein suitable for use as an oral vaccine. Production of such a protein in plants may be desirable, as plant protein production may be cost effective if yields are high.

Hennig et al. describe prior successful attempts to express recombinant OspA in chloroplasts of tobacco plant leaf cells with the use of a signal peptide from OspA, and the authors were also able to achieve successful transformation. However, those transgenic plants containing OspA in higher amounts (>1% total soluble protein (TSP)) were incapable of carrying out sufficient photosynthesis, and rapidly died unless sugars were exogenously applied. (FEBS J 274(21):5749-58 (2007).)

Accordingly, there is a need in the art for high-level expression of recombinant OspA proteins in plants, where the growth of the plant is not compromised, so as to be able to produce large amounts of the proteins over multiple generations. Such proteins should be suitable for inclusion in compositions for orally vaccinating an animal host, in order to break the transmission cycle of Lyme disease.

### SUMMARY OF THE INVENTION

The present invention relates to compositions and methods for vaccinating against Lyme disease and/or controlling the spread of Lyme disease, and particularly relates to compositions and methods for vaccinating wild animals against Lyme disease caused by exposure to Borrelia burgdorferi.

The methods of the present invention permit the affordable production of large amounts of recombinant OspA protein(s), which may then be used to vaccinate animals orally. The present invention therefore also relates to the production of OspA protein(s), and preferably to the recombinant production of OspA protein(s) in plant cells. The present invention also relates to recombinantly-produced OspA protein(s), and their use in vaccines against Lyme disease. There is an unmet need in the art for such compositions and methods.

The present invention meets the unmet needs in the art by providing methods for producing OspA protein(s) in plant cells, plant seeds containing OspA protein(s), compositions comprising recombinant OspA protein(s), and methods for vaccinating against Lyme disease and/or preventing the spread of Lyme disease by administering OspA protein(s), where said administration route is preferably oral.

One aspect of the invention comprises a method for vaccinating against Lyme disease, particularly Lyme disease caused by exposure to B. burgdorferi, by oral administration of at least one OspA protein, preferably a recombinant OspA protein.

An additional aspect of the invention is a method of vaccinating Lyme disease reservoirs utilizing economical large scale production of OspA in plants, such that the disease cycle is broken and vaccination of human subjects is not needed.

Another aspect of the invention comprises a pharmaceutical composition comprising at least one OspA protein formulated for oral administration. An additional aspect of the invention comprises a method of producing an OspA protein in plant seeds, comprising the steps of (a) transforming a plant cell with a chimeric gene comprising (i) a promoter derived from a gene encoding a seed-maturation-specific protein from a plant (i.e., a promoter from a plant gene that has upregulated activity during seed maturation), (ii) a first nucleic acid sequence, operably linked to the promoter, encoding a seed-maturation-specific signal sequence as a substitute for a signal peptide from OspA, and (iii) a second nucleic acid sequence, linked in translation frame with the first nucleic acid sequence, encoding an OspA protein, wherein the first nucleic acid sequence and the second nucleic acid sequence together encode a fusion protein comprising a signal sequence, preferably an N-terminal signal sequence, and the OspA protein; (b) producing a plant from the transformed plant cell and growing it for a time sufficient to produce seeds containing the OspA protein; and (c) harvesting the seeds from the plant.

According to a further aspect of the invention, a fertile and phenotypically normal plant is produced from the transformed plant cell of step (b), which is then grown in order to produce seeds containing the OspA protein.

An additional aspect of the invention comprises a method of transforming a plant cell, preferably a monocot plant such as rice, by incorporating a polynucleotide segment that encodes one or more OspA proteins.

Another aspect of the invention comprises administering to a subject that does not have Lyme disease a composition comprising one or more OspA proteins produced from plant cells, in order to produce immunity to B. burgdorferi, and prevent the subject from developing Lyme disease following subsequent exposure to B. burgdorferi.

Another aspect of the invention comprises a method of producing at least one OspA protein, the method comprising a) providing a plant cell transformed with a vector containing a promoter and a gene, operably linked to the promoter, encoding an OspA protein, b) producing a plant from the transformed plant cell and growing it for a time sufficient to produce seeds containing the OspA protein, c) harvesting the mature seeds, and optionally d) purifying the desired OspA protein from the seeds or seed product.

According to a further aspect of the invention, a fertile and phenotypically normal plant is produced from the transformed plant cell of step (b), which is then grown in order to produce seeds containing the OspA protein.

Another aspect of the invention relates to a chimeric gene comprising (i) a promoter that is active in plant cells; (ii) a first nucleic acid sequence, operably linked to the promoter, encoding a seed storage protein; and (iii) a second nucleic acid sequence, operably linked to the promoter, encoding an OspA protein; wherein the first and second nucleic acid sequences are linked in translation frame and together encode a fusion protein comprising the storage protein and the OspA protein.

A still further aspect of the invention relates to a vector comprising (i) a maturation-specific protein promoter from a monocot plant, (ii) a first DNA sequence, operably linked to the promoter, encoding a monocot plant seed-specific signal sequence, and (iii) a second DNA sequence, linked in translation frame with the first DNA sequence, encoding an OspA protein, wherein the first DNA sequence and the second DNA sequence together encode a fusion protein comprising a signal sequence, preferably an N-terminal signal sequence, and the OspA protein.

Yet another aspect of the invention comprises a method of producing seeds that express an OspA protein and a seed storage protein as a fusion partner, the method comprising (a) transforming a plant cell with a chimeric gene comprising: (i) a promoter that is active in plant cells; (ii) a first nucleic acid sequence, operably linked to the promoter, encoding a seed storage protein; (iii) a second nucleic acid sequence, operably linked to the promoter, encoding an OspA protein; and (iv) optionally a signal sequence, preferably a seed-specific signal sequence, wherein the first and second nucleic acid sequences and the optional signal sequence are linked in translation frame and together encode a fusion protein comprising the storage protein, the OspA protein and the optional signal sequence; (b) producing a plant from the transformed plant cell and growing it for a time sufficient to produce seeds containing the fusion protein; and (c) harvesting the seeds from the plant.

According to a further aspect of the invention, a fertile and phenotypically normal plant is produced from the transformed plant cell of step (b), which is then grown in order to produce seeds containing the OspA protein.

Other novel features and advantages of the present invention will become apparent to those skilled in the art upon examination of the following or upon learning by practice of the invention.

### BRIEF DESCRIPTION OF THE DRAWINGS

FIG. 1 shows the prediction of N-glycosylation sites in the OspA protein sequence. The asparagines predicted to be N-glycosylated are underlined, and the other amino acid residues in the conserved N-glycosylation site (Asn-Xaa-Ser/Thr) are in lower case and italics.

FIG. 2 shows the alignment of native OspA versus mutated and codon-optimized OspA. Original represents the native OspA gene sequence in the Borrelia burgdorferi. Codon-opt represents the OspA gene sequence with codon optimization and mutations at some potential N-glycosylation sites. Amino Acid represents the translated amino acid sequence corresponding to the codon-optimized OspA gene sequence. The underlined amino acid residues are different from their native form by mutation to abrogate the N-glycosylation sites. The numbers represent the position of nucleotides in the gene sequence. The vertical lines show the identity of nucleotides between the OspA gene sequence before and after codon optimization.

FIGS. 3A-3C are diagrams of three plasmid constructs:
VB15 (Gt1p::Gt1s::OspA::Nos).
VB16 (Chi26p::OspA::Nos), and
VB17 (Gt1p::Amys::OspA::Nos),
   where Gt1=glutelin-1 gene promoter; Chi26=barley chitinase gene promoter; Gt1SP=glutelin protein signal peptide; OspA=coding sequence of OspA gene codon-optimized based on rice codon usage preference; Nos=nopaline synthase terminator from Agrobacterium tumefaciens; and M=genetic codon for methionine amino acid.

FIGS. 4A-E are photographs which show the production of transgenic rice plants to express Lyme disease controlling gene OspA. A=embryonic calli induced from mature rice seeds; B and C=green plantlets regenerated from the hygromycine-resistant embryonic calli; D and E=transgenic plants regenerated from the root-induction medium.

FIGS. 5A-D are PCR analysis panels of regenerated plants from Hygromycin B-resistant calli. Panel A shows schematic representation of the gene constructs for expression of OspA in rice. Elements indicated are: Promoter, either rice glutelin gene (gt1) promoter or barley chitinase gene (CHI26) promoter; OspA, the cDNA encoding the OspA protein. The scale is not proportional to the physical size of each DNA fragment. Panels B, C, and D show representative PCR analysis of R0 transgenic plants generated from plasmid constructs VB15, VB16, and VB 17, respectively. On the top of each panel, H2O=the blank control of PCR reaction (negative control); −=the untransformed plant DNA (negative control); +=each respective plasmid vector DNA (positive control); M=1000-bp DNA ladder; and 1-22=transgenic plants generated from each respective plasmid vector. The arrow points to the expected PCR product (577 bp) amplified from the OspA gene sequence.

FIGS. 6A-C are photographs which show representative transgenic plants grown in greenhouse, where 6A shows transgenic plants derived from gene construct VB 15, 6B shows transgenic plants derived from gene construct VB 16, and 6C shows transgenic plants derived from gene construct VB 17.

FIG. 7 shows an expression analysis of individual R1 seeds through SDS-PAGE. M=protein molecular weight marker in kDa; Taipei309=non-transgenic rice cultivar; VB15-5-1 to 8=eight randomly selected R1 seeds of transgenic event VB15-5. 30 ul of protein extract from each seed was loaded on a 12% Tris-Glycine SDS-PAGE gel (Invitrogen), and the gel was stained with the Coomassie Blue solution. The arrowhead indicates the protein band corresponding to the recombinant OspA protein.

FIGS. 8A-B show a Western blot analysis for recombinant OspA protein expressed in rice transgenic plants. Panel A=Coomassie blue-stained SDS-PAGE gel; panel B=western blot probed with monoclonal anti-OspA antibody. The arrow indicates the protein band corresponding to the OspA protein. M=protein molecular weight marker in kDa; WT=non-transgenic rice cultivar Taipei309; Transgenic=protein extract from VB15-derived transgenic R1 seeds expressing recombinant OspA protein; B. burgdorferi=protein extract from bacteria B. burgdorferi. Protein samples were resolved on 12% Tris-glycine SDS-PAGE gels (Invitrogen) and transferred to nitrocellulose membrane using a Mini Trans-Blot Cell (Bio-Rad).

FIG. 9 shows the quantification of recombinant OspA protein expressed in rice grains. M=protein molecular weight marker in kDa; WT=non-transgenic rice cultivar Taipei309; VB15-5=a transgenic line expressing recombinant OspA protein; Soy trypsin inhibitor=standard protein purchased form Sigma with known concentration. Serial dilution of both VB15-5-derived protein extracts and protein standard soy trypsin inhibitor loaded on a 12% Tris-Glycine SDS-PAGE gels was indicated on top of each lane. The arrowhead indicates the protein band corresponding to native OspA protein. The estimate of the expression level of rOspA protein was made based on the comparison of intensity of the target bands against standard with the Kodak 1D image analysis program.

### DETAILED DESCRIPTION OF THE PRESENTLY PREFERRED EMBODIMENTS

The present invention relates to methods for the production of OspA proteins, preferably B. burgdorferi OspA proteins, and more preferably to methods for producing recombinant OspA proteins in plant cells. The present invention also relates to recombinantly-produced OspA proteins, and their use in oral vaccine formulations, particularly oral vaccine formulations for administration to animals. The present invention further relates to methods vaccinating against Lyme disease and/or preventing Lyme disease comprising orally administering at least one OspA protein, and to compositions for oral administration that comprise one or more OspA proteins. The compositions and method of the present invention may be beneficially utilized to vaccinate animals and/or humans in order to prevent them from contracting Lyme disease when exposed to B. burgdorferi or other agents that cause Lyme disease. When used in animals, these methods may remove a vector for the B. burgdorferi spp. that cause Lyme disease, thereby preventing their transmission to humans.

B. burgdorferi OspA proteins within the scope of the present invention may include those derived from B. burgdorferi sensu stricto S-1-10 and C-1-11, Borrelia afzelii BV1, Borrelia garinii LV4, B. afzelii PKo, B. valaisiana strains, B. burgdorferi sensu lato LV5, B. burgdorferi PKo, B. burgdorferi PBi, B. burgdorferi B31, B. burgdorferi ZS7, and B. burgdorferi N40, but are not limited to these. Any B. burgdorferi OspA proteins, including those yet to be identified, may be used in accordance with the compositions and methods of the present invention.

The open reading frame of the B. burgdorferi OspA gene consists of 822 nucleotides corresponding to a protein of 273 amino acids, including 16 amino acids as a signal peptide, and the protein has a calculated molecular mass of 29.6 kDa. High level expression of this protein in tobacco cells is lethal to the plant (see, e.g., FEBS J 274(21):5749-58 (2007)). The proteins contain a variable middle region, whereas the N and the C terminus are conserved. There is an unexpectedly high level of dissimilarity between the various OspA genes, and this may make it important to incorporate more than one OspA protein into a vaccine in order to confer optimum immunity.

### 1. DEFINITIONS

Unless otherwise indicated, all terms used herein have the meanings given below or are generally consistent with the same meaning that the terms have to those skilled in the art of the present invention. Practitioners are particularly directed to Sambrook et al., Molecular Cloning: A Laboratory Manual (Second Edition), Cold Spring Harbor Press, Plainview, N.Y. (1989); Ausubel F M et al., Current Protocols in Molecular Biology, John Wiley & Sons, New York, N.Y. (1993); and Gelvin and Schilperoot, eds. Plant Molecular Biology Manual, Kluwer Academic Publishers, The Netherlands (1997), for definitions and terms of the art.

General and specific techniques for producing proteins from plant cells may be obtained from the following applications, each of which is incorporated herein in its entirety by reference: U.S. patent application Ser. No. 09/847,232 (“Plant Transcription Factors and Enhanced Gene Expression”); U.S. patent application Ser. No. 10/077,381 (“Expression of Human Milk Proteins in Transgenic Plants”); U.S. patent application Ser. No. 10/411,395 (“Human Blood Proteins Expressed in Monocot Seeds”); U.S. patent application Ser. No. 10/639,779 (“Production of Human Growth Factors in Monocot Seeds”); U.S. patent application Ser. No. 10/639,781 (“Method of Making an Anti-infective Composition for Treating Oral Infections”); and international application no. PCT/US2004/041083 (“High-level Expression of Fusion Polypeptides in Plant Seeds Utilizing Seed-Storage Proteins as Fusion Carriers”).

The nucleic acids of the invention may be in the form of RNA or in the form of DNA, and include messenger RNA, synthetic RNA and DNA, cDNA, and genomic DNA. The DNA may be double-stranded or single-stranded, and if single-stranded may be the coding strand or the non-coding (anti-sense, complementary) strand.

By “host cell” is meant a cell containing a vector and supporting the replication and/or transcription and/or expression of the heterologous nucleic acid sequence. Preferably, according to the invention, the host cell is a plant cell. Other host cells may be used as secondary hosts, including bacterial, yeast, insect, amphibian or mammalian cells, to move DNA to a desired plant host cell.

A “plant cell” refers to any cell derived from a plant, including undifferentiated tissue (e.g., callus) as well as plant seeds, pollen, propagules, embryos, suspension cultures, meristematic regions, leaves, roots, shoots, gametophytes, sporophytes and microspores.

The term “mature plant” refers to a fully differentiated plant.

The term “seed” refers to all seed components, including, for example, the coleoptile and leaves, radicle and coleorhiza, scutulum, starchy endosperm, aleurone layer, pericarp and/or testa, either during seed maturation and seed germination. In the context of the present invention, the term “seed” and “grain” is used interchangeably.

The term “seed product” includes, but is not limited to, seed fractions such as de-hulled whole seed, flour (seed that has been de-hulled by milling and ground into a powder), a seed extract, preferably a protein extract (where the protein fraction of the flour has been separated from the carbohydrate fraction), malt (including malt extract or malt syrup) and/or a purified protein fraction derived from the transgenic grain.

The term “biological activity” refers to any biological activity typically attributed to that protein by those skilled in the art.

“Seed components” refers to carbohydrate, protein, and lipid components extractable from seeds, typically mature seeds.

“Seed maturation” refers to the period starting with fertilization in which metabolizable reserves, e.g., sugars, oligosaccharides, starch, phenolics, amino acids, and proteins, are deposited, with and without vacuole targeting, to various tissues in the seed (grain), e.g., endosperm, testa, aleurone layer, and scutellar epithelium, leading to grain enlargement, grain filling, and ending with grain desiccation.

“Maturation-specific protein promoter” refers to a promoter exhibiting substantially up-regulated activity (greater than 25%) during seed maturation.

“Heterologous nucleic acid” refers to nucleic acid which has been introduced into plant cells from another source, or which is from a plant source, including the same plant source, but which is under the control of a promoter that does not normally regulate expression of the heterologous nucleic acid.

“Heterologous peptide or polypeptide” is a peptide or polypeptide encoded by a heterologous nucleic acid. The peptides or polypeptides include OspA proteins, preferably B. burgdorferi OspA proteins. OspA proteins include, but are not limited to, those derived from B. burgdorferi sensu stricto S-1-10 and C-1-11, Borrelia afzelii BV1, Borrelia garinii LV4, B. afzelii PKo, B. valaisiana strains, B. burgdorferi sensu lato LV5, B. burgdorferi PKo, B. burgdorferi PBi, B. burgdorferi B31, B. burgdorferi ZS7, and B. burgdorferi N40. Any B. burgdorferi OspA proteins, including those yet to be identified, may be used in accordance with the compositions and methods of the present invention.

As used herein, the terms “native” or “wild-type” relative to a given cell, polypeptide, nucleic acid, trait or phenotype, refers to the form in which that is typically found in nature.

As used herein, the term “purifying” is used interchangeably with the term “isolating” and generally refers to any separation of a particular component from other components of the environment in which it is found or produced. For example, purifying a recombinant protein from plant cells in which it was produced typically means subjecting transgenic protein-containing plant material to separation techniques such as sedimentation, centrifugation, filtration, and chromatography. The results of any such purifying or isolating step(s) may still contain other components as long as the results have less of the other components (“contaminating components”) than before such purifying or isolating step(s).

As used herein, the terms “transformed” or “transgenic” with reference to a host cell means the host cell contains a non-native or heterologous or introduced nucleic acid sequence that is absent from the native host cell. Further, “stably transformed” in the context of the present invention means that the introduced nucleic acid sequence is maintained through two or more generations of the host, which is preferably (but not necessarily) due to integration of the introduced sequence into the host genome.

As used herein, the terms “reservoir” or “reservoir species” or “reservoir animal(s)” with reference to Lyme disease or B. burgdorferi means a non-human population that serves as a host for Lyme disease causing agents, particularly B. burgdorferi.

As used herein, the term “vector” with reference to the transmission of Lyme disease and the disease cycle refers to agents such as ticks that commonly transmit a Lyme disease causing agent from one host to another.

As used herein, the term “disease cycle” with reference to OspA refers to the process by which a vector, such as a tick, transmits a Lyme disease causing agent to a suitable host, such as a rodent. The host then transfers Lyme disease causing agents to other vectors, such as when ticks feed on the infected blood of rodents, thus completing the cycle.

As used herein, the term “excipients” with reference to a product containing OspA protein refers to any substance, not itself a therapeutic agent, which is used as a carrier or vehicle for delivery of the OspA. The excipients may include standard pharmaceutical excipients, and may also include any components that may be used to prepare foods and beverages for human and/or animal consumption, or bait formulations.

### 2. RECOMBINANT PRODUCTION OF OspA PROTEINS

The invention provides OspA proteins recombinantly produced in a host plant seed. Preferably, the OspA protein expressed comprises about 2% or greater of the total soluble protein in the seed. Thus, for example, the yield of total soluble protein which comprises the OspA protein targeted for production can be about 3% or greater, about 5% or greater, about 8% or greater, about 9% or greater, about 10% or greater, most preferably about 20% or greater, of the total soluble protein found in the recombinantly engineered plant seed.

Preferably, the OspA protein constitutes at least 0.01 weight percent in the harvested seeds. More preferably, the OspA protein constitutes at least 0.05 weight percent, most preferably at least 0.1 weight percent in the harvested seeds.

An embodiment of the present invention is a method of producing an OspA protein in plant seeds, comprising the steps of:

(a) transforming a plant cell with a chimeric gene comprising
(i) a promoter derived from a gene encoding a seed-maturation-specific protein from a plant,
(ii) a first nucleic acid sequence, operably linked to the promoter, encoding a seed-specific signal sequence as a substitute for the OspA signal peptide, and
(iii) a second nucleic acid sequence, linked in translation frame with the first nucleic acid sequence, encoding an OspA protein without its signal peptide, wherein the first nucleic acid sequence and the second nucleic acid sequence together encode a fusion protein comprising a signal sequence, preferably an N-terminal signal sequence, and the OspA protein;

(b) growing a plant from the transformed plant cell for a time sufficient to produce seeds containing the OspA protein; and

(c) harvesting the seeds from the plant.

Preferably, the plant is a monocot plant. More preferably, the plant is a cereal, preferably selected from the group consisting of rice, barley, wheat, oat, rye, corn, millet, triticale and sorghum.

Because the recombinant OspA protein(s) of the present invention are produced in plants, they may include plant glycosyl groups at one or more of the available glycosylation sites of the OspA protein(s). For example, a preferred embodiment of the invention relate to N-glycosylated OspA protein(s) produced in monocot seeds, such as rice, barley, wheat, oat, rye, corn, millet, triticale and sorghum. Most OspA proteins include five sites for N-glycosylation. When produced by the methods of the invention, the OspA protein(s) may be glycosylated at all five sites, at any four sites, at any three sites, at any two sites, or at any single glycosylation site. If a variant of OspA having a different number of glycosylation sites is utilized, it may be glycosylated at all or less than all of the glycosylation sites. It is also possibly to remove any or all of the plant glycosyl groups, if desired.

The promoter is preferably from a maturation-specific monocot plant storage protein or an aleurone- or embryo-specific monocot plant gene. Other promoters may be used, however, and the choice of a suitable promoter is within the skill of those in the art. More preferably, the promoter is a member selected from the group consisting of rice globulins, glutelins, oryzins and prolamines, barley hordeins, wheat gliadins and glutenins, maize zeins and glutelins, oat glutelins, sorghum kafirins, millet pennisetins, rye secalins, lipid transfer protein Ltp1, chitinase Chi26 and Em protein Emp1. Most preferably, the promoter is selected from the group consisting of rice globulin Glb promoter and rice glutelin Gt1 promoter.

The seed-specific signal sequence used to replace the signal peptide from OspA is preferably from a monocot plant, although other signal sequences may be utilized. Preferably, the monocot plant seed-specific signal sequence is associated with a gene selected from the group consisting of glutelins, prolamines, hordeins, gliadins, glutenins, zeins, albumin, globulin, ADP glucose pyrophosphorylase, starch synthase, branching enzyme, Em, and Iea. Most preferably, the monocot plant seed-specific signal sequence is a rice glutelin Gt1 signal sequence. Other monocot plant seed-specific signal sequence are associated with genes selected from the group consisting of α-amylase, protease, carboxypeptidase, endoprotease, ribonuclease, DNase/RNase, (1-3)-β-glucanase, (1-3)(1-4)-β-glucanase, esterase, acid phosphatase, pentosamine, endoxylanase, β-xylopyranosidase, arabinofuranosidase, β-glucosidase, (1-6)-β-glucanase, perioxidase, and lysophospholipase.

As will be understood by those of skill in the art, in some cases it may be advantageous to use a nucleotide sequences possessing non-naturally occurring codons. Codons preferred by a particular eukaryotic host can be selected, for example, to increase the rate of expression or to produce recombinant RNA transcripts having desirable properties, such as a longer half-life, than transcripts produced from naturally occurring sequence. As an example, it has been shown that codons for genes expressed in rice are rich in guanine (G) or cytosine (C) in the third codon position (Huang et al., J. CAASS 1: 73-86 (1990)). Changing low G+C content to a high G+C content has been found to increase the expression levels of foreign protein genes in barley grains (Horvath et al., Proc. Natl. Acad. Sci. USA 97: 1914-19, (2000)). If a rice plant is selected, the genes employed in the present invention may be based on the rice gene codon bias (Huang et al., supra) along with the appropriate restriction sites for gene cloning. These codon-optimized genes may be linked to regulatory and secretion sequences for seed-directed expression and these chimeric genes then inserted into the appropriate plant transformation vectors.

Another embodiment of the present invention is a method of producing seeds that express an OspA protein and a seed storage protein as a fusion partner, the method comprising:

(a) transforming a plant cell with a chimeric gene comprising:
(i) a promoter that is active in plant cells;
(ii) a first nucleic acid sequence, operably linked to the promoter, encoding a seed storage protein;
(iii) a second nucleic acid sequence, operably linked to the promoter, encoding an OspA protein; and
(iv) optionally a signal sequence, preferably a seed-specific signal sequence, wherein the first and second nucleic acid sequences and the optional signal sequence are linked in translation frame and together encode a fusion protein comprising the storage protein, the OspA protein, and the optional signal sequence;

(b) growing a plant from the transformed plant cell for a time sufficient to produce seeds containing the fusion protein; and

(c) harvesting the seeds from the plant.

The promoter and signal sequence may be selected from those discussed supra. The type of promoter and signal sequence is not critical to this embodiment of the invention. Preferably, the signal sequence targets the attached fusion protein to a location such as an intracellular compartment, such as an intracellular vacuole or other protein storage body, mitochondria, or endoplasmic reticulum, or extracellular space, following secretion from the host cell.

The seed storage protein is preferably from a monocot plant. Preferably, the seed storage protein is selected from the group consisting of rice globulins, rice glutelins, oryzins, prolamines, barley hordeins, wheat gliadins and glutenins, maize zeins and glutelins, oat glutelins, sorghum kafirins, millet pennisetins, or rye secalins. Rice globulin and rice glutelin are more preferred.

Suitable selectable markers for selection in plant cells include, but are not limited to, antibiotic resistance genes, such as kanamycin (nptII), G418, bleomycin, hygromycin, chloramphenicol, ampicillin, tetracycline, and the like. Additional selectable markers include a bar gene which codes for bialaphos resistance; a mutant EPSP synthase gene which encodes glyphosate resistance; a nitrilase gene which confers resistance to bromoxynil; a mutant acetolactate synthase gene (ALS) which confers imidazolinone or sulphonylurea resistance. The particular marker gene employed is one which allows for selection of transformed cells as compared to cells lacking the nucleic acid which has been introduced. Preferably, the selectable marker gene is one that facilitates selection at the tissue culture stage, e.g., an nptII, hygromycin or ampicillin resistance gene. Thus, the particular marker employed is not essential to this invention.

In general, a selected nucleic acid sequence is inserted into an appropriate restriction endonuclease site or sites in the vector. Standard methods for cutting, ligating and E. coli transformation, known to those of skill in the art, are used in constructing vectors for use in the present invention.

Plant cells or tissues are transformed with above expression constructs using a variety of standard techniques. It is preferred that the vector sequences be stably integrated into the host genome. Particularly preferred plants are those that have been transformed with an OspA gene, or have been grown from a plant cell that has been transformed with an OspA gene, in accordance with the methods described above, and express an OspA protein as a result of the transformation. Still more preferred are plants that have been transformed with an OspA gene, or have been grown from a plant cell that has been transformed with an OspA gene, that are fertile and phenotypically normal and express an OspA protein.

According to another aspect of the invention, plants that have been transformed with the OspA gene exhibit growth that is comparable to a wild-type plant of the same species, or exhibit fertility that is comparable to a wild-type plant of the same species, or both. A transformed plant that exhibits comparable growth to a wild-type plant preferably produces at least 80% of the amount of total biomass produced by a wild-type plant grown under similar conditions, such as location (e.g., greenhouse, field, etc.), soil type, nutrients, water, and exposure to sunlight. Preferably, the transformed plant produces at least 85%, more preferably at least 90%, and still more preferably at least 95% of the amount of total biomass produced by a wild-type plant grown under similar conditions. A transformed plant that exhibits comparable fertility to a wild-type plant preferably produces at least 80% of the amount of offspring produced by a wild-type plant grown under similar conditions, such as location (e.g., greenhouse, field, etc.), soil type, nutrients, water, and exposure to sunlight. Preferably, the transformed plant produces at least 85%, more preferably at least 90%, and still more preferably at least 95% of the amount of offspring produced by a wild-type plant grown under similar conditions.

According to a further aspect of the invention, the plants transformed with the OspA gene that are comparable to a wild-type plant of the same species also express the OspA protein as a result of the transformation. Preferably the transformed plants express the OspA protein at high levels, e.g., 2%, 3%, 5%, 8%, 9%, 10%, or 20% or greater of the total soluble protein in the seeds of the plant.

The method used for transformation of host plant cells is not critical to the present invention. For commercialization of the heterologous peptide or polypeptide expressed in accordance with the present invention, the transformation of the plant is preferably permanent, i.e., by integration of the introduced expression constructs into the host plant genome, so that the introduced constructs are passed onto successive plant generations. The skilled artisan will recognize that a wide variety of transformation techniques exist in the art, and new techniques are continually becoming available.

Any technique that is suitable for the target host plant may be employed within the scope of the present invention. For example, the constructs can be introduced in a variety of forms including, but not limited to, as a strand of DNA, in a plasmid, or in an artificial chromosome. The introduction of the constructs into the target plant cells can be accomplished by a variety of techniques, including, but not limited to calcium-phosphate-DNA co-precipitation, electroporation, microinjection, Agrobacterium-mediated transformation, liposome-mediated transformation, protoplast fusion or microprojectile bombardment. The skilled artisan can refer to the literature for details and select suitable techniques for use in the methods of the present invention.

Transformed plant cells are screened for the ability to be cultured in selective media having a threshold concentration of a selective agent. Plant cells that grow on or in the selective media are typically transferred to a fresh supply of the same media and cultured again. The explants are then cultured under regeneration conditions to produce regenerated plant shoots. After shoots form, the shoots can be transferred to a selective rooting medium to provide a complete plantlet. The plantlet may then be grown to provide seed, cuttings, or the like for propagating the transformed plants.

The expression of the heterologous peptide or polypeptide may be confirmed using standard analytical techniques such as Western blot, ELISA, PCR, HPLC, NMR, or mass spectroscopy, together with assays for a biological activity specific to the particular protein being expressed.

The invention also includes a chimeric gene, comprising:

(i) a promoter that is active in plant cells;

(ii) a first nucleic acid sequence, operably linked to the promoter, encoding a seed storage protein; and

(iii) a second nucleic acid sequence, operably linked to the promoter, encoding an OspA protein;

wherein the first and second nucleic acid sequences are linked in translation frame and together encode a fusion protein comprising the storage protein and the OspA protein.

The seed storage protein may be at the N-terminal or C-terminal side of the OspA protein in the fusion protein. It is preferred that the seed storage protein be located at the N-terminal side of the OspA protein.

The fusion protein may also be engineered to comprise at least one selective purification tag and/or at least one specific protease cleavage site for eventual release of the OspA protein from the seed storage protein fusion partner, fused in translation frame between the OspA protein and the seed storage protein. Preferably, the specific protease cleavage site may comprise enterokinase (ek), Factor Xa, thrombin, V8 protease, Genenase™, α-lytic protease or tobacco etch virus (TEV) protease. The fusion protein may also be cleaved chemically.

### 3. DOSAGE FORMS CONTAINING ONE OR MORE OspA PROTEINS

Oral dosage forms are preferred for administering the OspA protein(s) produced in accordance with the present invention due to their ease of administration; however, parenteral formulations containing the recombinant OspA protein(s) of the present invention are also envisioned and these may be prepared in accordance with known methods.

Because the recombinant OspA protein(s) of the present invention are produced in plants, they may include plant glycosyl groups at one or more of the available N-glycosylation sites of the OspA protein(s). For example, a preferred embodiment of the invention relate to glycosylated OspA protein(s) produced in monocot seeds, such as rice, barley, wheat, oat, rye, corn, millet, triticale and sorghum. Most OspA proteins include five sites for glycosylation. When produced by the methods of the invention, the OspA protein(s) may be glycosylated at all five sites, at any four sites, at any three sites, at any two sites, or at any single glycosylation site. If a variant of OspA having a different number of N-glycosylation sites is utilized, it may be glycosylated at all or less than all of the N-glycosylation sites. Optionally, any or all plant glycosyl groups may be removed.

The oral formulations according to the present invention can be prepared in any manner suitable to deliver the OspA protein(s). Examples of dosage forms for administration to a human include a tablet, a caplet, a hard or soft capsule, a lozenge, a cachet, a dispensable powder, granules, a suspension or solution, an elixir, a liquid, or any other form reasonably adapted for oral administration. Examples of dosage forms for administration to an animal include foods, liquids, baits, and any other compositions that are likely to be consumed by the animal to be vaccinated.

To help the release of OspA protein(s) in small intestine, the oral formulations may be tableted or pelleted, or encapsulated, and preferably enteric-coated. Enteric coating prevents a tablet or capsule from dissolving before it reaches the small intestine. Alternatively the material may be spheronized into microparticles and preferably enterically coated. Spheroids may be produced in the size range of 250 μm to 850 μm. Enteric coatings are known to be selectively insoluble substances that do not dissolve in the acidic environment of the stomach, but dissolve in the higher pH of the small intestine, resulting in a specific release of OspA protein(s) in the small intestine.

The one or more OspA proteins can be further formulated together with one or more pharmaceutically acceptable excipients to produce a pharmaceutical composition. The term “excipient” herein means any substance, not itself a therapeutic agent, used as a carrier or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients include, by way of illustration and not limitation, diluents, disintegrants, binding agents, adhesives, wetting agents, lubricants, glidants, crystallization inhibitors, surface modifying agents, substances added to mask or counteract a disagreeable taste or odor, flavors, dyes, fragrances, and substances added to improve appearance of the composition.

Excipients employed in compositions of the invention can be solids, semi-solids, liquids or combinations thereof. Compositions of the invention containing excipients can be prepared by any known technique of pharmacy that comprises admixing an excipient with a drug or therapeutic agent.

Other excipients such as colorants, flavors, and sweeteners, which may make the oral formulations of the present invention more desirable to animal hosts of B. burgdorferi tick vectors can also be used in compositions of the present invention.

The oral formulations containing OspA protein(s) can be prepared by any suitable process, not limited to processes described herein. Conventional blending, tableting, and encapsulation techniques known in the art can be employed.

### 4. ORAL VACCINES AGAINST LYME DISEASE COMPRISING RECOMBINANT OspA PROTEIN(S)

The present invention provides compositions and methods for orally administering Lyme disease vaccines to subject animals or humans, to prevent the subjects from developing Lyme disease after exposure to Lyme disease-causing agents, particularly B. burgdorferi spp. The methods include administering oral vaccine formulations containing one or more OspA protein(s), preferably one or more recombinant OspA protein(s) that have been prepared in accordance with the present invention. The oral formulations may be prepared using monocot seeds, such as rice, barley, wheat, oat, rye, corn, millet, triticale and sorghum, where the seeds have been genetically-modified to express OspA protein(s). A presently preferred monocot seed is rice.

The oral vaccine formulations according to the present invention can be provided in any manner suitable for delivering a dose of OspA protein capable of inducing an immune response in the organism that consumes the formulation. According to one aspect of the invention, oral vaccine formulations including more than one type of OspA protein may be provided. This approach is believed to be beneficial in conferring immunity against Lyme disease-causing agents, particularly B. burgdorferi spp., because it may induce the production of a variety of different antibodies. The oral formulations for vaccinating against Lyme disease may include recombinant OspA proteins derived from one or more of B. burgdorferi sensu stricto S-1-10 and C-1-11, Borrelia afzelii BV1, Borrelia garinii LV4, B. afzelii PKo, B. valaisiana strains, B. burgdorferi sensu lato LV5, B. burgdorferi PKo, B. burgdorferi PBi, B. burgdorferi B31, B. burgdorferi ZS7, and B. burgdorferi N40, but they are not limited to these strains. Any B. burgdorferi OspA proteins, including those yet to be identified, may be used in the oral vaccine formulations of the present invention.

When oral formulations are prepared from a genetically-modified monocot seed, it is possible to first purify the recombinant OspA protein(s), and then incorporate them into a food, beverage, or bait formulation. In accordance with this aspect of the invention, any components that are added to the genetically-modified monocot seed to form a food, beverage, or bait formulation may be considered excipients. One of the benefits of the present invention is the ability to directly utilize the genetically-modified monocot seed in the production of such a food, beverage, or bait formulations without first purifying the OspA protein(s). This is possible at least in part because of the relatively high levels of the recombinant OspA protein(s) in the seeds produced by the methods of the present invention.

The oral formulations containing OspA protein(s) according to the present invention may be administered in any dose adequate to vaccinate an animal, i.e., induce an immune response in said animal to OspA protein(s), thereby preventing the animal from contracting Lyme disease in the event of future exposure to Lyme disease-causing agents, particularly B. burgdorferi spp. This in turn prevents the spread of Lyme disease to other animals or humans, by preventing or eliminating the presence of Lyme disease causing agents from vectors that feed upon the infected animal, particularly ticks. In one embodiment of the present invention, the oral formulation is administered in doses of from about 1 mg/day to about 10 g/day, preferably 5 mg/day to 5 g/day, more preferably 25 mg/day to 2.5 g/day.

According to other preferred embodiments, it is also possible to prepare parenterally-administered vaccines for Lyme disease using the recombinant OspA protein(s) produced in monocot seeds by first purifying the OspA protein(s) from the seeds, and then incorporating them into a standard parenteral vaccine formulation using techniques known in the art. Such parenteral vaccines may be administered in any amount sufficient to confer immunity to Lyme disease-causing agents, particularly B. burgdorferi spp.

### EXAMPLES

### Overview

Three plasmids named VB15, VB16 and VB17, which were designed to express recombinant OspA protein in protein bodies within endosperm cells, aleurone layer, embryo, and extracellular space, respectively, were prepared with codon-optimized nucleotide sequences for mature OspA protein. Two rice cultivars, Tapei309 and Bengal, were transformed with these three gene constructs by biolistic particle bombardment of embryonic calli induced from the mature seeds. The transgenic plants carrying the OspA gene expression cassette were confirmed through PCR amplification of genomic DNA isolated from the regenerated plants using primers specific to the OspA gene. More than 200 transgenic plants were produced from each of VB15, VB16, and VB17 gene constructs, and over 100 transgenic plants from each construct set transgenic seeds. Expression analysis of transgenic rice seeds identified positive transgenic events expressing recombinant OspA protein. The recombinant OspA protein has been proved to be recognized by monoclonal anti-OspA antibody, and the expression level of recombinant OspA protein in one of the best transgenic events is 8.8% of total soluble protein and 0.1% of rice seed weight. OspA expressed in rice grain is soluble, and the expression level is high. The positive transgenic rice plants expressing OspA protein have been advanced to the R4 generation, and exhibit normal growth and fertility as compared with non-transgenic plants in both the greenhouse and the field.

### Synthesis of OspA Gene

Mutation of N-Glycosylation Sites Present in OspA Protein Sequence

It is known that the proteins expressed in higher plants undergo different post-translational modifications (PTMs) compared to the bacteria system. The N-glycosylation is the major PTM that can potentially alter the protein structure and thus affect the effectiveness of OspA as an antigen.

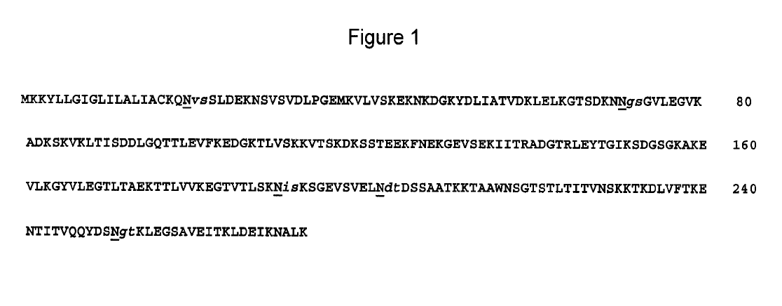

Five potential N-glycosylation sites, at Asparagine (N) residues 20, 71, 190, 202, and 251 were identified in the OspA amino acid sequence (FIG. 1). The three C-terminus N-glycosylation sites at N residues 251, 202, and 190 could be more likely to adversely perturb the correct OspA conformation structure (personal communication with Dr. Johnson at CDC), and mutations to abrogate the N-glycosylation at these sites were performed. To abrogate these N-glycosylation sites, the naturally occurring mutations in Borrelia species were extrapolated (if they exist). At the N-glycosylation sites 251 (NGT) and 190 (NISK), an Alanine (A) residue occurs at the position of amino acid N residue in Eurasian Lyme disease Borrelia and the position of amino acid Serine (S) residue in B. afzelii, respectively (personal communication with Dr. Johnson), and thus the substitute of N or S to A at these two N-glycosylation sites was carried out. Considering the structure similarity between N-glycosylation sites 190 and 202, A was substituted for the Threonine (T) at the N-glycosylation site 202 (NDT).

Codon-Usage Optimization and Synthesis of OspA Gene

As the coding gene sequences in the rice genome are rich in guanines (G) or cytosines (C) in the third position of genetic codons, expression of heterologous genes with codon optimization according to the preference of rice genes could lead to high level of heterologous protein expression. The codons for the above mutated mature OspA protein were optimized based on the codon-usage preference of rice genes after rare codons were eliminated and other potentially unfavorable features associated with codons were taken into consideration to avoid.

To facilitate the cloning of OspA gene into plasmid vectors, the MlyI blunt-cutting restriction site that allows cut immediately before the first nucleotide of OspA gene was engineered upstream the OspA gene. The XhoI and the Sad restriction sites were engineered right after the stop codon of OspA gene. The entire nucleotide sequence was synthesized through BlueHeron, cloned into a pUC119-derived plasmid vector pUC57, and verified through sequencing on both orientations. The resulting plasmid is designated as pUC-OspA.

### Plasmid Constructs for Rice Transformation

To express the OspA gene in rice grain with potentially different protein trafficking and targeting routes, three OspA expression vectors named VB15, VB16, and VB17 were made (FIG. 3).

VB15 (Gt1p+Gt1s+OspA) to lead recombinant OspA protein to be deposited into protein bodies in seed endosperm cells. Gt1p is the promoter of rice glutelin-1 gene, and the promoter is developmentally regulated and only active in rice grain endosperm. Gt1s is the signal sequence of the glutelin 1 gene to lead the protein to the protein body in the endosperm cell.

The backbone vector to make plasmid construct VB15 is Ventria's existing plasmid pAPI405, which contains the Gt1p promoter and its signal peptide (Gt1s), the β-glucuronidase (Gus) gene, and the NOS terminator. The Gus gene fragment in pAPI 405 was removed by double digestion with restriction enzymes NaeI and XhoI, and then replaced with the MlyI-XhoI fragment of OspA gene from plasmid pUC-OspA by in-frame ligation. The correctness of plasmid construct was verified via sequencing, and the plasmid was designated as VB15 (FIG. 3A).

VB16 (Chi26p+OspA) for expression of OspA in embryo and aleurone layer. Chi26p is the promoter of barley chitinase gene (GenBank accession number BLYCHI26).

The backbone vector to make plasmid construct VB16 is Ventria's existing plasmid pAPI217, which contains the barley chitinase 26 promoter, the Gus gene, and the NOS terminator. The pAPI217 plasmid was first opened up by XbaI, which is immediately downstream to the Chi26 promoter, followed by being blunted with Mung bean nuclease, and then cut by SacI (upstream the Nos terminator) followed by dephosphorylation with calf intestinal alkaline phosphatase (CIAP).

To express the OspA gene for mature OspA protein driven directly under the Chi26 promoter, a start genetic codon (ATG) for methionine amino acid before the OspA gene is required. The ATG codon was engineered by site-directed mutagenesis method using pUC-OspA plasmid as template, and the resulting plasmid was designated as pUC-M-OspA. The OspA gene with the added start codon ATG was released from pUC-M-OspA by MlyI and SacI double digestion, and inserted underneath the Chi26 promoter by in-frame ligation. The correctness of plasmid construct was verified via sequencing, and the plasmid was designated as VB16 (FIG. 3B).

VB17 (Gt1p+Amys+OspA) for secretion of OspA to outer space Amys is the signal sequence of the barley amylase gene (GenBank protein ID AAA98790.1) and functions as a leader for protein secretion through the cell membrane.

The PCR-based mutation approach was used with the pVB15 plasmid as the template to replace the Gt1 signal peptide with the Amylase peptide.

The pair of PCR primers is designed as follows:

Forward:
5′ CTTGGCCTGTCGGCCAGCTTGGCCTCCGGGCAATGCAAGCAGAACGT
TTCTAG 3′
Reverse:
5′ GAGGACGAGGAAGAGGGAGAGGGACAAGTGTTTGTTTGCCATGTTGT
TGTAGGAC 3′

The nucleotides italicized in the forward primer and reverse primer matched to the starting region of OspA gene and the ending region of Gt1 promoter in the VB15 plasmid, respectively. Both primers were phosphorylated at the 5′ end. The PCR reaction was carried out in 50 ul of reaction containing 10 ng of VB15 plasmid DNA, 0.2 mM each dNTP, 200 pmol of each primer, 5 μl of 10×Pfu buffer, 1 unit of Pfu DNA polymerase (Stratagene). The PCR amplification conditions was 94° C., 3 min followed by 25 cycles of 94° C., 40 s; 56° C., 1 min; and 72° C. 8 min. The PCR product was then gel purified, ligated, and transformed into DH10B cells by electroporation. The correctness of plasmid construct was verified via sequencing, and the plasmid was designated as VB17 (FIG. 3C).

In addition, plasmid pAPI146 was also used in transformation to provide selection markers. The pAPI146 plasmid consists of the hpt (hygromycin B phosphor-transferase) gene encoding the hygromycin B-resistant protein under the control of a rice beta-glucanase 9 gene promoter, which can restrict the expression of the hpt gene only in rice calli.

For each plasmid construct, the linear DNA fragments containing the minimal expression cassette were cut out from the plasmid DNA to exclude any vector backbone sequence.

### Generation of Transgenic Plants

Two rice cultivars, Tapei 309 and Bengal, were used for gene transformation. Rice seeds were dehusked, sterilized in 20% (v/v) commercial bleach for 20 min, and washed with sterile water three times for five min each. The sterilized seeds were placed on rice callus induction (RCI) medium containing N6 salt (Sigma), B5 vitamins (Sigma), 2 mg/L 2,4-D, and 3% sucrose for 10 days to induce callus. Then the primary callus was sub-cultured on RCI medium for two weeks.

The particle bombardment transformation was performed with the Biolistic PDS-1000/He system (Bio-Rad). Prior to transformation, the 2-4 mm diameter calli were selected and placed in a 4 cm diameter circle on RCI medium with 0.3 M mannitol and 0.3 M sorbitol for 24 h. Then the calli were bombarded with 1.5 mg of gold particles (60 μg/μl) coated with 2.5 μg of expression cassette DNAs for expression of hygromycin B (selectable marker gene) and OspA gene, respectively, at a ratio of 1 to 2 at a helium pressure of 1100 psi. After bombardment, the calli were recovered on the same medium plate for 48 h, and then transferred to RCI medium with 80 mg/L hygromycin B to incubate in the dark at 26° C. for 45 days to select the hygromycin B-resistant calli.

The hygromycin B-resistant transformants were selected and transferred to RCI medium (without 2,4-D) with 5 mg/L ABA, 2 mg/L BAP, 1 mg/L NAA for 9 to 12 days in dim light followed by being transferred onto the regeneration medium consisting of RCI medium without 2,4-D, 3 mg/l BAP, and 0.5 mg/l NAA and cultured under continuous lighting conditions for two to four weeks. After the regenerated plants were 1 to 3 cm high, the plantlets were transferred to rooting medium containing the half concentration of the MS medium (Sigma) plus 1% sucrose and 0.05 mg/l NAA for two weeks to allow the development of roots.

### Polymerase Chain Reaction (PCR) Analysis of Transgenic Plants

The PCR analysis of the regenerated plants was conducted using the Extract-N-Amp Plant PCR kit (Sigma). The pair of PCR primers were: OspA-F: CCCAGGCGAAATGAAAGTTC-3′ and OspA-R: 5′-TGTGATAGTGAGGGTTGAGG-3′, which are located near the start and end region of OspA gene, respectively. The amplification reaction condition was as follows: 94° C. for 5 min, 35 cycles of 94° C., 1 min; 60° C. for 1 min; and 72° C. for 1 min followed by 72° C. for 10 min. The PCR products were resolved in 1.2% agarose gel by electrophoresis.

### Plant Culture in Greenhouse

The PCR-confirmed transgenic plants were then transferred to a 6.5×6.5 cm pot containing a mix of 50% commercial soil, Sunshine #1 (Sun Gro Horticulture Inc, WA) and 50% natural soil from rice fields, and nursed in growth chamber under continuous light and high humidity (nearly 100%) for one week. Then, the plants were transplanted into 8 inch pots and transferred to a closed greenhouse, where the temperatures were maintained at 30° C. during day time and 25° C. during night time and supplemental light were added. Fertilizer, water and pest management were carried out according to good agricultural practice to ensure healthy growth of the transgenic rice plants.

### Expression Analysis of Recombinant OspA in Transgenic R1 Seeds

To screen the expression of OspA in rice grains, eight R1 seeds from each transgenic event were randomly picked, dehusked, and put into eight wells of one column of a 96 deep-well plate. 500 μl of extraction buffer (PBS, pH 7.4) was added into each well containing seed and soaked for 3 h at room temperature followed by adding two 10 mm diameter of steel beads per well and vortexing with Geno/Grinder 2000 (SPEX CertiPrep, Metuchen, N.J.) for 20 min at 1300 strokes/min. Then the mixture was centrifuged at 4,000 rpm for 20 min at 4° C. in a microplate-centrifuge (Eppendorf), and the crude protein extract was transferred to a new microplate. Equal amount of protein extracts from each of eight seeds of one transgenic event was pooled, and resolved on 12% Tris-glycine SDS-PAGE gels for expression screening analysis. For VB16- and VB17-derived transgenic events, seeds were also screened with the PBS buffer (pH 7.4) plus 2% sarkosyl and 1% triton x-114, as the PBS buffer alone failed to show the potential target protein band on Coomassie blue-stained SDS-PAGE gels.

To verify the recombinant OspA protein identified through SDS-PAGE gel, immunoblot detection of OspA was performed. The protein extracts were resolved on 12% Tris-glycine SDS-PAGE gel, then transferred onto nitrocellulose membrane at 100 V, 350 mA for 1 hr using a Mini Trans-Blot Cell (Bio-Rad) containing transfer buffer of 25 mM Tris pH 8.3, 192 mM glycine, 20% (v/v) methanol. The blot was blocked for 1 hr in blocking buffer (KPL Laboratories) at RT, and incubated in monoclonal anti-OspA antibody (a gift from Dr. Johnson, CDC) at a 1:1000 dilution in blocking buffer for 2 h at room temperature. The blot was then washed four times, each 5 minutes, with TBST (25 mM Tris pH 7.4, 135 mM NaCl, 0.05% Tween), and then incubated with secondary antibody (anti-mouse AP conjugate) at a concentration of 1:2000 in TBST for 45 minutes at room temperature followed by washing with TBST four times, for 5 minutes each time. The blot was incubated with BCIP substrate (Sigma) for 10 minutes to develop.

To estimate the expression level of recombinant OspA in transgenic rice seeds, the protein extracts of the selected transgenic seeds were resolved on the SDS-PAGE in parallel with the titration of the known amount of trypsin protein inhibitor (Sigma). Then the amount of target protein was estimated by comparing the intensity of the target protein band with that of trypsin protein inhibitor using the Kodak 1D image analysis program.

### Results

### Genetic Manipulation of N-Glycosylation Sites and Codon Usage of OspA Protein

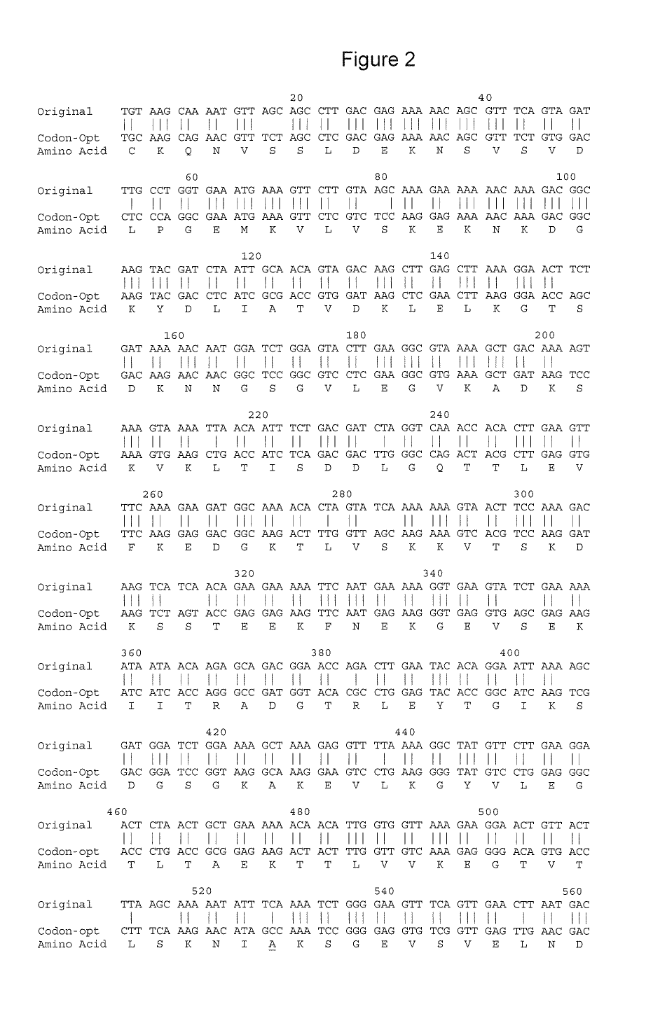

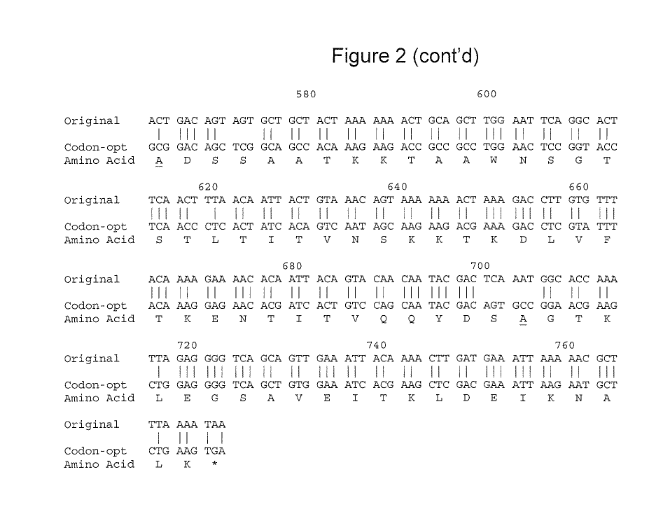

As proteins expressed in plants and bacteria including B. burgdorferi undergo different N-glycosylations, and the bulky N-glycosylation may perturb the OspA protein conformational structure and affect the effectiveness of recombinant OspA as antigen, three amino acid residues at three C-terminal N-glycosylation sites (Asn-Xaa-Ser/Thr) were altered to abrogate these potential N-glycosylations (FIG. 1). On the other hand, to obtain high level expression of OspA protein in rice plants, genetic codons for OspA protein were adapted to the preference of rice genes before gene synthesis. In the optimized OspA gene sequence, 189 out of 258 codons for mature OspA protein were changed compared to its original gene sequence, and the G+C content in the optimized sequence was increased to 52.3% from 34.0% in the original sequence (FIG. 2).

### OspA Gene Expression Constructs

Three expression constructs were made to express the mature OspA in rice grains with different protein targeting (FIG. 3). In plasmid vector VB15, gene sequence for mature OspA was fused in-frame under the drive of glutelin-1 gene promoter and its signal peptide (FIG. 3A). The combination of glutelin-1 gene promoter and its signal peptide not only leads high expression level of recombinant proteins but could also direct majority of proteins to be deposited into protein bodies (Yang et al., 2003). The latter can contribute to protection of OspA protein from protease degradation and also relates to high level expression. However, it is unknown about to what extent the protein will be lipidated in protein bodies. Both lipidated and non-lipidated OspA had been shown to have immunological effects against B. burgdorferi (Tsao et al., 2004). However, some studies show that lipidated OspA protein might be more effective as an antigen than the non-lipidated OspA (Johnson et al., 1995).

To make the recombinant OspA protein more likely to be lipidated, the glutelin signal peptide in the VB15 plasmid vector was replaced with a rice amylase signal peptide to direct OspA to the outer membrane (FIG. 3C). In addition, considering that high level of lipid is accumulated in rice grain embryo and aleurone layer cells, and protein lipidation should occur more likely in these cells than in other type of cells, a third plasmid vector was made to fuse the gene for the mature OspA protein under the drive of a chitinase promoter, which has been proved to express gene explicitly embryo and aleurone layer cells (FIG. 3B).

### Rice Transformation and Regeneration of Transgenic Plants

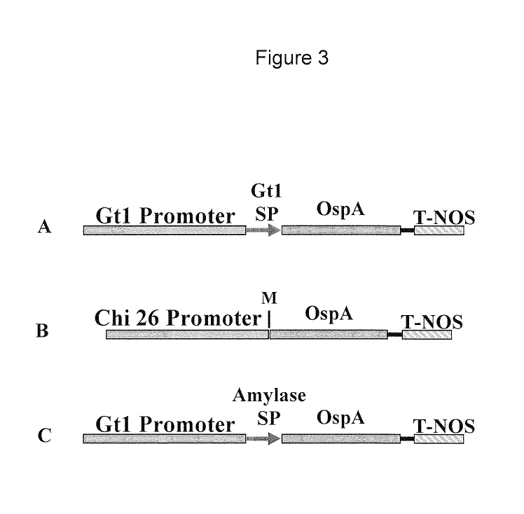

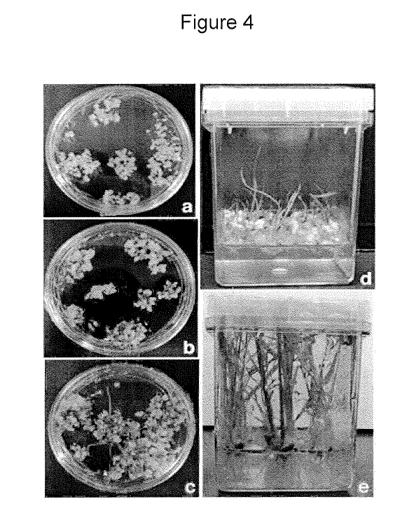

From two rice cultivars, Taipei309 and Bengal, large quantities of embryonic calli (FIG. 4a) were induced with our described procedure. After bombardment transformation, about 50% of calli survived on the medium containing the Hygromycin B, indicating that the transformation efficiency was about 50% in terms of expression of Hygromycin B. Most of the Hygromycin B-resistant calli (90%) were able to regenerate plantlets on the regeneration medium (FIGS. 4b and c), and at least 214 regenerated plants (FIGS. 4d and e) were produced from each of the three gene constructs used for transformation (Table 1).

TABLE 1
Number of transgenic plants derived from different gene
constructs and rice cultivar

| | | No. of | | Co-trans- | No. of |
|---|---|---|---|---|---|
| | | independent | No. of | formation | transgenic |
| | Rice | plants from | OspA+ | efficiency | plants in |
| Constructs | cultivar | Hyg^{R} calli | plants | (%)* | GH |
| VB15 | Bengal | 58 | 58 | 100 | 58 |
| | Taipei309 | 169 | 156 | 92 | 156 |
| VB16 | Bengal | 103 | 93 | 90 | 93 |
| | Taipei309 | 265 | 252 | 95 | 252 |
| VB17 | Bengal | 56 | 50 | 88 | 50 |
| | Taipei309 | 252 | 219 | 87 | 219 |

*the co-transformation efficiency = (no. of plants showing the presence of OspA gene/no, of independent plants from Hyg^{R} calli) × 100

Selection of the Regenerated Plants with the OspA Gene

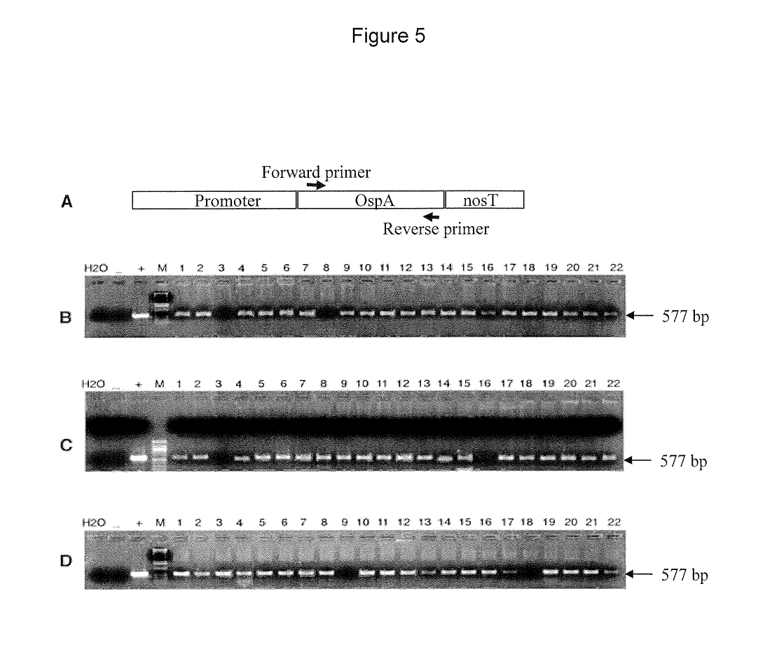

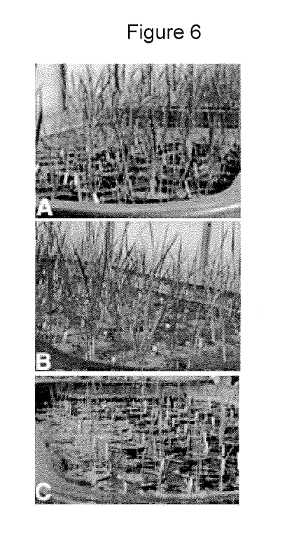

The selection marker gene and target gene can be co-transformed but with a wide range of efficiency in different studies. To confirm the insertion of OspA expression cassette DNA into the rice genome and eliminate the escape plants (without the OspA gene), a PCR analysis of regenerated plants was conducted with primers specific to OspA gene (FIG. 5A.). The PCR amplification revealed a band with the expected size in most of the transgenic plants but not in the wild-type plants (FIGS. 5B-D). According to the PCR results for all transgenic plants, the percentage of plants with OspA gene out of the plants from Hygromycine-resistant calli, also called co-transformation efficiency, was from 87% to 100% in different combinations of gene construct and rice cultivar (Table 1). All the PCR-verified transgenic plants (R0) from each gene construct were transferred to a closed greenhouse to set the seeds.

Expression Analysis of the Regenerated Plants with the OspA Gene

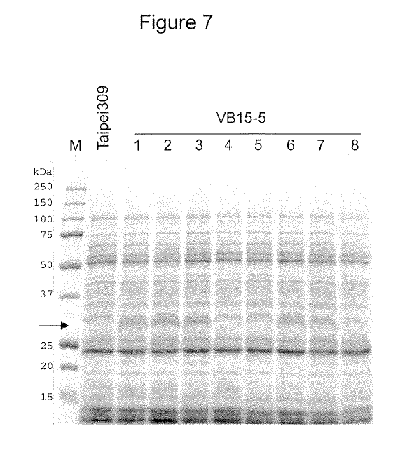

The SDS-PAGE analysis was used to screen R1 seeds of transgenic events. The number of transgenic events for expression analysis was summarized in Table 2. As segregation of the inserted transgene and its expression for recombinant OspA protein would be anticipated in R1 seeds, eight pooled R1 seeds from each transgenic line were used for SDS-PAGE protein assay as a first quick screen to identify the positive events. In VB15-derived R1 seeds, the PBS extraction buffer (pH=7.4) extracted a protein corresponding to the size of native OspA protein (about 28 kDa) in some transgenic seeds while this protein species was absent in the non-transgenic control (FIG. 7). In both VB16- and VB17-derived R1 seeds, however, PBS extraction buffer failed to identify a similar protein band as revealed in VB15-derived seeds. With the addition of detergents sarkosyl and Triton X-114 to PBS buffer, a protein band corresponding to the expected size of native OspA protein was shown in some VB16 and VB17 transgenic R1 seeds but absent in wild-type rice seeds. It is noted that the intensity of this potential target protein band in VB16 and VB17 seeds is much weaker than that in VB15 R1 seeds.

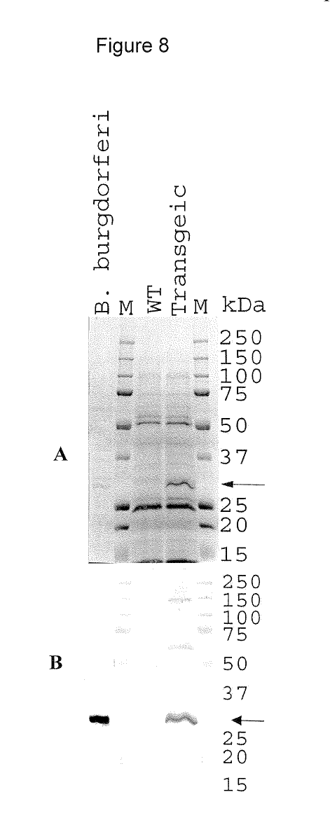

In order to verify the authenticity of the possible OspA protein identified through SDS-PAGE in the transgenic events but absent in the non-transgenic rice, western blot analysis using monoclonal anti-OspA antibody was performed (FIG. 8). The immunoblot showed three distinct bands in VB15 R1 seeds, but none were shown in the non-transgenic rice (FIG. 8B). Furthermore, the three immunobands correspond to a monomer, dimer, and pentamer of native OspA protein. These multimers of OspA protein shown in the blot were not likely to be due to the incomplete denaturing of protein prior to SDS-PAGE gel, as repeated and harsher denaturing conditions still could not break down the multimer into monomer (data not shown). In addition, a close look at the position of monomer-OspA protein showed additional faint bands above the predominant monomer protein band (FIG. 8B). A plausible explanation of these close bands could be that several isoforms of OspA co-existed due to the post-translational modifications of the OspA protein including N-glycosylation and lipidation.

It is noteworthy to point out that the amount of OspA protein in B. burgdorferi crude protein extract is much less than the recombinant OspA protein in rice seed crude protein extract according to the band intensity on the Coomassie blue-stained SDS-PAGE gel (FIG. 8A). However, the immuno-blot showed the opposite as seen in the Coomassie gel, as the less amount of OspA in B. burgdorferi showed stronger immuno-band compared to rice seed extract (FIG. 8B). It suggests that the monoclonal anti-OspA antibody used in this study has lower binding affinity to recombinant OspA from rice seed than the native OspA protein. This binding affinity difference could be explained by the fact that the monoclonal anti-OspA antibody that was used was raised against the native and N-glycosylated OspA protein, but the recombinant OspA protein had been manipulated to abrogate three C-terminal N-glycosylation sites.

The immunoblot hybridization with monoclonal anti-OspA antibody as probe showed only a faint immuno-band corresponding to native OspA protein in some VB16 and VB17 transgenic events but not in wild-type seeds.

### Estimation of Expression Level of Recombinant OspA

Out of 122 VB15 transgenic events, 34 were positive events expressing recombinant OspA protein (Table 2). 42 and 11 putative positive events were identified from 158 VB16 and 132 VB17 transgenic events, respectively, according to the Coomassie blue-stained SDS-PAGE analysis (Table 2).

TABLE 2
Summary of transgenic events expressing OspA protein

| | | | No. of | No. of |
|---|---|---|---|---|
| | | No. of | transgenic | OspA- |
| Plasmid | | transgenic | events | expressing |
| construct | Rice cultivar | events | with seeds | events |
| VB15 | Bengal | 58 | 10 | 4 |
| | TP309 | 156 | 112 | 30 |
| VB16 | Bengal | 93 | 21 | 11 |
| | TP309 | 252 | 137 | 31 |
| VB17 | Bengal | 50 | 17 | 6 |
| | TP309 | 219 | 115 | 5 |

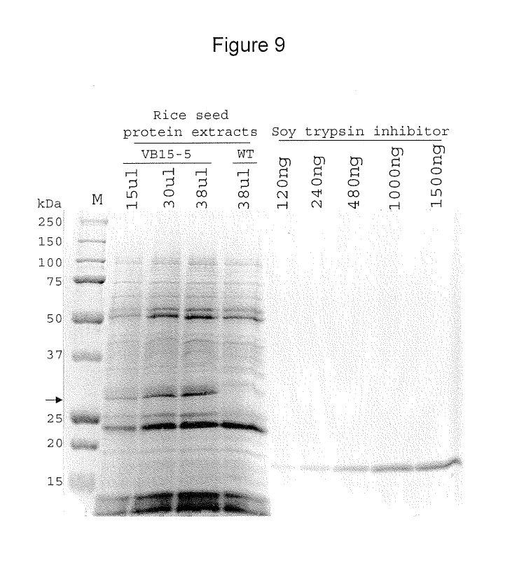

As the OspA was seen as one of the dominant bands in some VB15 R1 seeds extract on SDS-PAGE gel, its expression level was directly estimated on the SDS-PAGE gel using densitometry analysis with purified trypsin protein inhibitor as standard (FIG. 9). Expression levels were shown different between different events. The expression level of OspA in one of the best events, VB15-5 was estimated to be 8.8% of total soluble protein or 0.1% (1 mg/g⁻¹) of dehusked rice grain weight. This estimated expression level of OspA could be underestimated since the dimer and pentamer forms of OspA protein revealed in immunoblot were not seen in the SDS-PAGE gel and thus were not taken into account in the quantification of recombinant OspA.

The low level expression of OspA in VB16-derived transgenic seeds is as expected due to the small portion of embryo and aleurone layer cells in the seed grain. As for the VB17 plasmid construct, the low level expression could be due to the outer membrane trafficking and targeting of OspA protein.

Six transgenic events with high expression of OspA protein were selected (Table 3). The expression level of recombinant OspA protein in these selected events was within a range of 6 to 8.8% of total soluble protein, and 0.07% to 0.1% of seed weight.

TABLE 3
The selected transgenic events expressing recombinant OspA

| | | Estimated | Estimated |
|---|---|---|---|
| Transgenic | Source of rice | expression level | Percent Total |
| event IDs | cultivar | (% seed weight) | Soluble Protein |
| VB15-5 | Taipei309 | 0.1% | 8.8% |
| VB15-26 | Taipei309 | 0.09% | 8.0% |
| VB15-111 | Taipei309 | 0.07% | 6.5% |
| VB15-117 | Taipei309 | 0.07% | 6.0% |
| VB15-230 | Taipei309 | 0.09% | 7.9% |
| VB15-231 | Taipei309 | 0.1% | 8.8% |

A portion of the R1 seeds from each selected event were planted in greenhouse to produce seeds at R2 generation, and homozygous transgenic lines expressing OspA protein were selected. The selected homozygous R2 lines were advanced to next generations by being grown in both greenhouse and field, and the transgenic rice plants from generation to generation maintained the similar expression level of OspA while exhibiting both normal growth and full fertility as non-transgenic plants (Table 4).

TABLE 4
The expression level of OspA and major agronomic characteristics
of rice transgenic plants at different generations

| | | Estimated | Estimated | Plant | | | Seed |
|---|---|---|---|---|---|---|---|
| | Gene | expression level | expression level | height | No. of | No. of | weight |
| Plant ID | ration | (% seed weight) | (% total soluble protein) | (cm) | tillers | panicles | (mg/seed) |
| VB15-111 | R1 | 0.1% | 8.8% | 101 | 11 | 9 | 24.1 |
| VB15-111-4 | R2 | 0.1% | 9.0% | 102 | 10 | 10 | 24.3 |
| VB15-111-4 | R3 | 0.11% | 9.1% | 104 | 9 | 8 | 25.1 |
| VB15-111-4 | R4 | 0.11% | 9.0% | 102 | 8 | 8 | 25.0 |
| Non-transgenic | | 0 | 0 | 102 | 10 | 10 | 25.0 |
| TP309 | | | | | | | |

### CONCLUSION

The recombinant OspA protein was expressed in rice grains at the level of 8.8% total soluble protein, or 0.1% seed weight. More importantly, the recombinant OspA has been shown to be recognized by the monoclonal anti-OspA antibody, suggesting that recombinant OspA expressed in rice grains can potentially be used to develop an oral vaccine to break the disease transmission cycle. This expression level of recombinant OspA enables us to affordably produce large quantities of OspA to vaccinate animal reservoirs for controlling Lyme disease.

It will, of course, be appreciated that the above description has been given by way of example only and that modifications in detail may be made within the scope of the present invention.

Throughout this application, various patents and publications have been cited. The disclosures of these patents and publications in their entireties are hereby incorporated by reference into this application, in order to more fully describe the state of the art to which this invention pertains.

The invention is capable of considerable modification, alteration, and equivalents in form and function, as will occur to those ordinarily skilled in the pertinent arts having the benefit of this disclosure.

While the present invention has been described for what are presently considered the preferred embodiments, the invention is not so limited. To the contrary, the invention is intended to cover various modifications and equivalent arrangements included within the spirit and scope of the detailed description provided above.

1. A mature transgenic monocot seed from a fertile and phenotypically normal plant, wherein the seed expresses OspA.

2. The seed of claim 1, wherein the OspA is derived from a Borrelia spp. selected from the group consisting of B. burgdorferi sensu stricto S-1-10 and C-1-11, Borrelia afzelii BV1, Borrelia garinii LV4, B. afzelii PKo, B. valaisiana strains, B. burgdorferi sensu lato LV5, B. burgdorferi PKo, B. burgdorferi PBi, B. burgdorferi B31, B. burgdorferi ZS7, and B. burgdorferi N40.

3. The seed of claim 1, whose total soluble protein fraction contains at least 0.3% by total soluble protein weight of OspA.

4. The seed of claim 1 which is a mature, transgenic seed selected from the group consisting of rice, corn, barley, and wheat seeds.

5. (canceled)

6. A method of producing OspA in plant seeds, comprising the steps of:
(a) transforming a monocot plant cell with a chimeric gene comprising
(i) a promoter from a gene encoding a seed-specific protein from a plant,
(ii) a first nucleic acid sequence, operably linked to the promoter, encoding a signal sequence from a seed-specific protein as a substitute for an OspA signal peptide, and
(iii) a second nucleic acid sequence, linked in translation frame with the first nucleic acid sequence, encoding an OspA without its signal peptide, wherein the first nucleic acid sequence and the second nucleic acid sequence together encode a fusion protein comprising a signal sequence and the OspA;
(b) producing a fertile and phenotypically normal plant from the transformed monocot plant cell and growing it for a time sufficient to produce seeds containing the OspA; and
(c) harvesting the seeds from the plant.

7.-10. (canceled)

11. The method of claim 6, wherein the OspA comprises about 2% or greater of the total soluble protein in the seeds.

12. The method of claim 6, wherein the OspA comprises about 3% or greater of the total soluble protein in the seeds.

13. The method of claim 6, wherein the OspA is derived from a Borrelia spp. selected from the group consisting of B. burgdorferi sensu stricto S-1-10 and C-1-11, Borrelia afzelii BV1, Borrelia garinii LV4, B. afzelii PKo, B. valaisiana strains, B. burgdorferi sensu lato LV5, B. burgdorferi PKo, B. burgdorferi PBi, B. burgdorferi B31, B. burgdorferi ZS7, and B. burgdorferi N40.

14. A method of producing at least one OspA, comprising the steps of:
a) providing a monocot plant cell transformed with a vector containing a promoter and a gene, operably linked to the promoter, encoding an OspA,
b) producing a fertile and phenotypically normal plant from the transformed monocot plant cell and growing it for a time sufficient to produce seeds,
c) harvesting the mature seeds, and optionally
d) purifying the desired OspA from the seeds or seed product.

15. (canceled)

16. The method of claim 14, wherein the plant is a cereal selected from the group consisting of rice, barley, wheat, oat, rye, corn, millet, triticale and sorghum.

17. The method of claim 16, wherein the plant is rice.

18.-19. (canceled)

20. A vector, comprising (i) a promoter from a monocot plant gene that has upregulated activity during seed maturation, (ii) an optional first DNA sequence, operably linked to said promoter, encoding a monocot plant seed-specific signal sequence, and (iii) a second DNA sequence, linked in translation frame with the first DNA sequence, encoding an OspA, wherein the first DNA sequence and the second DNA sequence together encode a fusion protein comprising a signal sequence and the OspA.

21.-23. (canceled)

24. A method of administering one or more OspA proteins to an animal, comprising the step of orally administering to said animal an oral formulation comprising a composition made from the seed of claim 1.

25. The method of claim 24, wherein the one or more OspA proteins are administered as a vaccine for Lyme disease.

26.-30. (canceled)

31. An oral vaccine composition comprising at least one OspA protein and one or more excipients formulated for oral administration.

32. The oral vaccine composition of claim 31, wherein the at least one OspA protein is derived from a Borrelia spp. selected from the group consisting of B. burgdorferi sensu stricto S-1-10 and C-1-11, Borrelia afzelii BV1, Borrelia garinii LV4, B. afzelii PKo, B. valaisiana strains, B. burgdorferi sensu lato LV5, B. burgdorferi PKo, B. burgdorferi PBi, B. burgdorferi B31, B. burgdorferi ZS7, and B. burgdorferi N40.

33. The oral vaccine composition of claim 31, wherein the formulation is provided in a form selected from the group consisting of a tablet, caplet, hard capsule, soft capsule, lozenge, cachet, powder, granules, suspension, solution, elixir, liquid, beverage, and food.

34. A method of breaking a Lyme disease cycle by controlling pathogen prevalence in reservoir animals, comprising the steps of:
a) producing OspA in monocot seeds;
b) formulating OspA from monocot seeds into a reservoir-targeting oral vaccine formulation without extracting OspA from said monocot seeds;
c) administering the formulation to Lyme disease reservoirs to induce immunity in reservoir species, thus reducing pathogen levels in reservoir animals and associated vectors.

35. (canceled)

36. An oral vaccine produced by combining the OspA protein made by the method of claim 14 with one or more excipients.
